(19) United States
(12) Patent Application Publication
(10) Pub. No.: US 2022/0184433 A1
(43) Pub. Date: Jun. 16, 2022

(54) AIR TREATMENT SYSTEM

(71) Applicant: Microchip Technology Incorporated, Chandler, AZ (US)

(72) Inventor: Keith Edwin Curtis, Queen Creek, AZ (US)

(73) Assignee: Microchip Technology Incorporated, Chandler, AZ (US)

(21) Appl. No.: 17/501,449

(22) Filed: Oct. 14, 2021

Related U.S. Application Data
(60) Provisional application No. 63/123,523, filed on Dec. 10, 2020.

(51) Int. Cl. A62B 23/02; A61L 9/20; A62B 9/00; A62B 18/00; A62B 7/10; B01D 45/16; B04C 9/00; B04C 11/00; B03C 3/15; B03C 3/68; B01D 53/00; B01D 53/44; B01D 53/34; B01D 46/00; B01D 50/00; F24F 8/192; F24F 8/22

(52) U.S. Cl. CPC A62B 23/02; B01D 2259/4541; A62B 9/00; A62B 18/006; A62B 7/10; B01D 45/16; B04C 9/00; B04C 11/00; B03C 3/15; B03C 3/68; B01D 53/007; B01D 53/44; B01D 53/346; B01D 46/0032; B01D 50/002; F24F 8/192; F24F 8/22; A61L 2209/16; A61L 2209/14; A61L 2209/12; A61L 2209/111; B04C 2009/001; B04C 2009/002; B04C 2009/005; B01D 2258/06; B01D 2259/804; B01D 2259/4508; B01D 2259/4533; A61L 9/20

(57) ABSTRACT

An air treatment system includes a cyclone filter and an electrostatic filtration system. The cyclone filter may include a cyclone chamber, a cyclone chamber inlet configured to receive air including suspended particulates, and a cyclone chamber outlet configured to output treated air toward a respiratory interface, e.g., a mask or face shield. The cyclone filter produces a rotational airflow that removes at least some particulates from the air in the cyclone filter. The electrostatic filtration system is configured to charge the particulates in the cyclone chamber with a first polarity to produce an electrostatic attraction of the particulates to a particulate removal system charged with an opposite second polarity, to remove additional particulates from the cyclone filter. The air treatment system may also include an ultraviolet purification system to deliver ultraviolet radiation (e.g., UVC radiation) to kill, destroy or otherwise affect organic particulates in the air being treated.

### RELATED APPLICATION

This application claims priority to commonly owned U.S. Provisional Patent Application No. 63/123,523 filed Dec. 10, 2020, the entire contents of which are hereby incorporated by reference for all purposes.

### TECHNICAL FIELD

The present disclosure relates to air treatment systems, and more particularly, to air treatment systems for providing treated air for breathing.

### BACKGROUND

Systems for filtering or purifying breathing air include masks and other devices that filter airborne contaminants from the environment. Many masks utilize a passive filter, for example a cloth or cellulose filter. However, such passive filters typically provide limited virucide properties. A passive filter may become clogged with use, which increasingly restricts airflow through the filter. The clogged filter may also present a health risk to the wearer, as it may contain viral and/or bacterial contaminants. Some masks allow replacement of the passive filter, although this may be a difficult task. Replacement of the passive filter may require a stock of replacement filters, which may be expensive, and may also require frequent disposal of hazardous organic waste (used filters).

There is a need for a filter system that is effective at removing contaminants, for example organic and inorganic particulates, to provide treated air for breathing.

### SUMMARY

An air treatment system is provided for separating and removing a mass of particulates from a volume of air to produce treated air for breathing. In some examples, the air treatment system may deliver treated air to or toward a person's respiratory system, e.g., via a mask or a face shield. As another example, the air treatment system may deliver treated air to a room or other environment that may be occupied by one or more person or animal.

As used herein, “particulates” refers to any solid and/or liquid particles suspended in air, which may include organic and/or inorganic particles, for example dust, pollen, mold spores, soot, smoke, salt particles, liquid droplets, bacteria, viral particles, or any other types of particles, and may include particles of any size, including coarse particles, fine particles, and/or ultrafine particles. Further, as used herein, “treated air” means air treated by the air treatment system, which treatment may include (a) separation and removal of particulates from the air and/or (b) treatment of particulates by an ultraviolet purification system, as discussed below.

In some examples, the air treatment system combines multiple components and systems for separating and removing particulates from air. For example, the air treatment system may include a cyclone filter combined with an electrostatic filtration system for separating and removing particulates from a volume of air. In general, the cyclone filter removes larger particulates from the air, while the electrostatic filtration system utilizes electrostatic attraction to remove smaller particulates. The cyclone filter may include a cyclone chamber configured to receive air through an inlet and produce a rotational (cyclonic) flow inside the cyclone chamber that separates at least some particulates (especially larger particulates) from the air, which separated particulates may be transferred to a particulate removal system. For example, the separated particulates may fall into a particulate reservoir at the bottom of the cyclone chamber.

The electrostatic filtration system may augment the performance of the cyclone filter, e.g., by facilitating the separation and removal of particulates from the cyclone chamber, especially small particulates (e.g., having a diameter below 100 microns, below 20 microns, or even below 10 microns, for example including particulates with diameters in the range of 2.5-10 microns). Although certain bacteria and viruses have diameters below about 2 microns (e.g., many viruses have a diameter in the range of 20-500 nanometers), the cyclone filter and electrostatic filtration system may effectively remove water droplets and dust particles that carry bacteria and viruses.

In some examples, the electrostatic filtration system operates by (a) applying a first electric charge having a first polarity (e.g., high voltage negative charge) to conductive surface(s) of the cyclone chamber, to thereby charge particulates in the cyclone chamber with the first electric charge, and (b) applying a second electric charge having the opposite polarity of the first electric charge to conductive surface(s) of the particulate removal system (e.g., conductive surface(s) of a particulate reservoir at the bottom of the cyclone chamber). When air enters the cyclone chamber, the conductive surface(s) of the cyclone chamber impart the first polarity (e.g., negative charge) on the particulates in the air, such that the particulates are electrostatically repelled from the cyclone chamber walls and electrostatically attracts and pulled toward the particulate removal system (e.g., particulate reservoir) charged with the opposite second polarity (e.g., positive charge). An inlet and outlet of the cyclone chamber may also be charged with the first polarity to repel the charged particulates, such that the particulates are electrostatically attracted only to the particulate removal system (e.g., particulate reservoir).

In some examples, interior surface(s) of the cyclone chamber may be lined with a conductive material having anti-microbial/anti-virus properties, e.g., silver, copper, or a copper alloy (e.g., brass or bronze). In other examples, the interior surface(s) of the cyclone chamber may be lined with any other suitable conductive material, for example lead, tin, molybdenum, zirconium, zinc, stainless steel, nickel, cobalt, or titanium.

In addition, some examples include an ultraviolet purification system configured to deliver ultraviolet radiation to the cyclone chamber to affect organic particulates present in the cyclone chamber. For example, the ultraviolet purification system may deliver ultraviolet C (UVC) radiation to the cyclone chamber to kill, destroy, or otherwise modify virus and/or bacteria particulates in the cyclone filter. In one example, the UVC purification system may deliver UVC radiation having a wavelength in the range of 200-280 nm into the cyclone chamber. The cyclone chamber may be lined with a highly reflective material, e.g., silver or stainless steel, to promote reflection or scattering of UVC radiation in the cyclone chamber.

Further, some examples including a pressure-based control system including at least one pressure sensor and control electronics to dynamically control the operation of the air treatment system based on pressure measurements from the pressure sensor(s), e.g., to reduce power consumption and thereby extend battery life. The air treatment system may be configured to detect inhalation events and/or exhalation events, and selectively activate, deactivate, or otherwise control the operation of the electrostatic filtration system and/or ultraviolet purification system based on the detected inhalation events and/or exhalation events. For example, the air treatment system may be configured to activate the electrostatic filtration system and/or ultraviolet purification system only while a user is actively using the air treatment system for breathing, e.g., by automatically activating such system(s) upon a pressure-based detection of a user inhalation event and deactivating such systems after a defined period of no detected inhalation events. As another example, the air treatment system may be configured to activate the electrostatic filtration system and/or ultraviolet purification system only during the inhalation phase of each breath.

Airflow through the cyclone filter (i.e., through the cyclone chamber) may be supplied in any suitable manner, e.g., continuously, intermittently, or otherwise. For example, in some examples, the cyclone filter may be configured to deliver a pressurized flow of treated air to a user via a mask or face shield, e.g., using a blower (e.g., fan) or other device to provide a continuous or intermittent positive pressure air flow through the cyclone filter and to the user. In other examples, the air flow through the cyclone filter may be provided by user's respiration, e.g., where the system includes a sealed facemask. When the user inhales, a negative pressure is created, which draws a quantity of air into the cyclone filter, wherein particulates are removed and/or treated by ultraviolet radiation (e.g., UVC radiation), and delivers the treated air to the user via the facemask.

One aspect provides an air treatment system including a cyclone filter and electrostatic filtration system. The cyclone filter includes a cyclone chamber, an inlet configured to receive air including particulates into the cyclone chamber, an outlet configured to output treated air from the cyclone chamber, wherein the cyclone filter is configured to facilitate a rotational airflow within the cyclone chamber to remove at least a portion of the particulates from the received air, and a particulate removal system configured to receive particulates removed from the received air by the cyclone filter. The electrostatic filtration system comprising electrostatic filtration system electronics configured to apply a first electric charge having a first polarity to the particulates in the cyclone chamber and apply a second electric charge having a second polarity opposite the first polarity to the particulate removal system, such that the particulates in the cyclone chamber become charged with the first polarity and are electrostatically attracted to the particulate removal system.

In one example, the electrostatic filtration system electronics are configured to apply the first electric charge having the first polarity to at least one conductive surface of the cyclone chamber to thereby apply the first electric charge to the particulates in the cyclone chamber, and apply the second electric charge having the second polarity opposite the first polarity to at least one conductive surface of the particulate removal system.

In one example, at least one conductive surface of the cyclone chamber comprises silver or copper.

In one example, the air treatment system further includes a pressure sensor configured to monitor an air pressure, and pressure-based control electronics configured to dynamically control the electrostatic filtration system as a function of the monitored air pressure. In one example, the pressure-based control electronics are configured to dynamically control, based on the monitored air pressure, at least one of (a) a first voltage having the first polarity to at least one conductive surface of the cyclone chamber or (b) a second voltage having the second polarity to at least one conductive surface of the particulate removal system.

In one example, the pressure-based control electronics are configured to automatically detect inhalation events based on the monitored air pressure, the automatically detected inhalation events including at least one of a start of inhalation, an end of inhalation, or an occurrence of an inhalation, and automatically control the electrostatic filtration system based on the detected inhalation events.

In one example, the pressure-based control electronics are configured to automatically detect an inhalation event based on the monitored air pressure, automatically activate the electrostatic filtration system in response to the detected inhalation event, automatically detect a no-inhalation period during which no inhalation event is detected for a defined no-inhalation threshold duration, and automatically deactivate the electrostatic filtration system in response to the detected no-inhalation period.

In one example, the control electronics are configured to detect a start of inhalation by a user based on the monitored air pressure, activate the electrostatic filtration system as a function of the detected start of inhalation, detect an end of inhalation by the user based on the monitored air pressure, and deactivate the electrostatic filtration system as a function of the detected end of inhalation.

In one example, the air treatment includes an ultraviolet purification system configured to deliver ultraviolet radiation to the cyclone chamber to affect at least some of the particulates in the received air. In one example, the ultraviolet purification system is configured to deliver ultraviolet C (UVC) radiation to the cyclone chamber to affect organic particulates in the cyclone chamber. In one example, the air treatment includes a pressure sensor configured to monitor an air pressure, and pressure-based control electronics configured to control at least one of the electrostatic filtration system or the ultraviolet purification system as a function of the monitored air pressure. In one example, the pressure-based control electronics are configured to dynamically control the delivery of ultraviolet radiation to the cyclone chamber based on the monitored air pressure.

In one example, the air treatment system includes a pressure sensor configured to monitor an air pressure, and pressure-based control electronics configured to automatically detect inhalation events based on the monitored air pressure, the automatically detected inhalation events including at least one of a start of inhalation, an end of inhalation, or an occurrence of an inhalation, and automatically control the ultraviolet purification system based on the detected inhalation events.

In one example, the pressure-based control electronics are configured to automatically detect an inhalation event based on the monitored air pressure, automatically activate the ultraviolet purification system in response to the detected inhalation event, automatically detect a no-inhalation period during which no inhalation event is detected for a defined no-inhalation threshold duration, and automatically deactivate the ultraviolet purification system in response to the detected no-inhalation period.

In one example, the air treatment system includes a pressure sensor configured to monitor an air pressure, and pressure-based control electronics configured to detect a start of inhalation by a user based on the monitored air pressure, activate the ultraviolet purification system as a function of the detected start of inhalation, detect an end of inhalation by the user based on the monitored air pressure, and deactivate the ultraviolet purification system as a function of the detected end of inhalation.

In one example, the particulate removal system comprises a particulate repository configured to receive and store particulates removed from the cyclone chamber.

In one example, the air treatment system includes a replaceable filter arranged downstream of the cyclone filter outlet. In one example, the replaceable filter comprises a cloth or cellulose filter cartridge.

In one example, the air treatment system includes a blower configured to generate a positive pressure airflow through the cyclone filter.

In one example, the air treatment system includes a respiratory interface configured to connect the cyclone filter with a user's respiratory system so that the rotational airflow in the cyclone chamber is generated by an inhalation of the user. In one example, the respiratory interface comprises a facemask.

In one example, the air treatment system is a self-contained wearable system.

In one example, the air treatment system is configured for connection to a heating, ventilation, and air conditioning (HVAC) system.

In one example, the cyclone filter is configured to produce a rotational airflow in the cyclone chamber to propel at least a portion of the particulates in the cyclone chamber radially outwardly, resulting in particulate clusters that fall downwardly toward the particulate removal system.

Another aspect provides an air treatment system for treating contaminated air. The air treatment system includes a cyclone filter configured to receive air including particulates and produce a rotational airflow for removing at least some of the particulates, an ultraviolet purification system configured to deliver ultraviolet radiation to the cyclone filter to kill or destroy organic particulates included in the particulates in the cyclone filter, and an electrostatic filtration system configured to electrically charge the particulates in the cyclone filter to facilitate removal of particulates from the cyclone filter by electrostatic forces.

In one example, the electrostatic filtration system comprises electronics configured to apply a first electric charge having a first polarity to the particulates in the cyclone filter and apply a second electric charge having a second polarity opposite the first polarity to a particulate removal system, such that the particulates in the cyclone filter become charged with the first polarity and are electrostatically attracted to the particulate removal system.

### BRIEF DESCRIPTION OF THE DRAWINGS

Example aspects of the present disclosure are described below in conjunction with the figures, in which:

FIG. 1 shows an example air treatment system including a cyclone filter and an electrostatic filtration system;

FIG. 2 shows an example air treatment system including a cyclone filter, an electrostatic filtration system, and a pressure-based control system;

FIG. 3 shows an example air treatment system including a cyclone filter, an electrostatic filtration system, and an ultraviolet purification system;

FIG. 4 shows an example air treatment system including a cyclone filter, an electrostatic filtration system, an ultraviolet purification system, and a pressure-based control system;

FIG. 5A shows an example air treatment system connected with a heating ventilation and air conditioning (HVAC) system for recycling treated air to a room;

FIG. 5B shows an example air treatment system connected with a heating ventilation and air conditioning (HVAC) system for providing treated air to a room; and

FIG. 5C shows an example air treatment system connected with a heating ventilation and air conditioning (HVAC) system for delivering treated air out of a room containing contaminated air.

It should be understood that the reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

An air treatment system is provided for removing particulates from a volume of air to produce treated air for breathing. The air treatment system may include a cyclone filter and an electrostatic filtration system. The cyclone filter may include a cyclone chamber, a cyclone chamber inlet configured to receive air including suspended particulates, and a cyclone chamber outlet configured to output treated air toward a respiratory interface, e.g., a mask or face shield. The cyclone filter produces a rotational airflow that removes at least some particulates from the air in the cyclone filter. The electrostatic filtration system may be configured to charge the particulates in the cyclone chamber with a first polarity to produce an electrostatic attraction of the particulates to a particulate removal system charged with an opposite second polarity, to remove additional particulates from the cyclone filter. The air treatment system may also include an ultraviolet purification system to deliver ultraviolet radiation (e.g., UVC radiation) to kill, destroy or otherwise affect organic particulates in the air being treated.

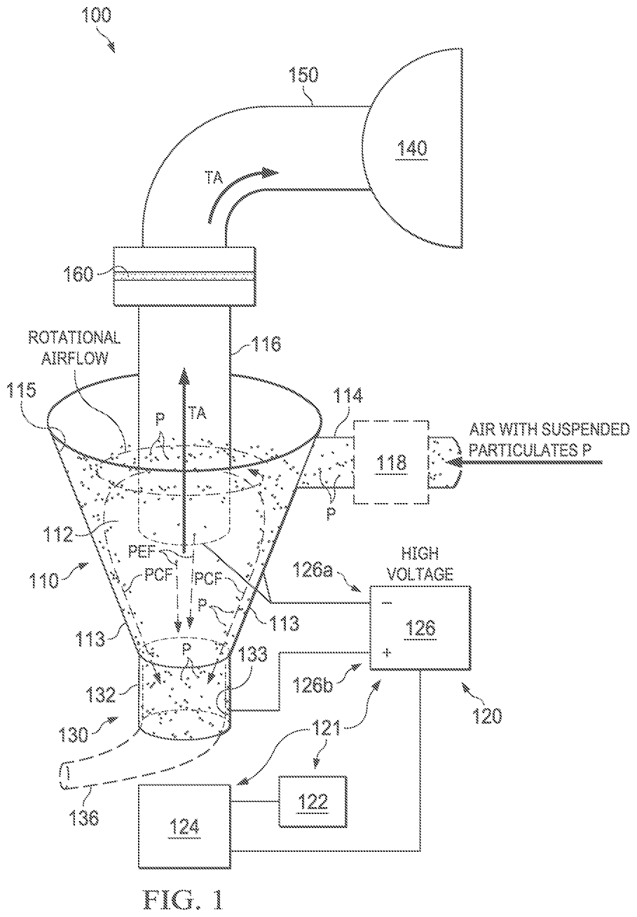

FIG. 1 shows an example air treatment system 100. Air treatment system 100 includes a cyclone filter 110, an electrostatic filtration system 120, and a particulate removal system 130. The cyclone filter 110 is generally operable to remove particulates P from a volume of air to be treated, which particulates P are delivered to the particulate removal system 130. The electrostatic filtration system 120 is operable to generate electrostatic forces to further facilitate the removal of particulates P from the cyclone filter 110, as discussed below.

The cyclone filter 110 includes a cyclone chamber 112, an inlet 114 and an outlet 116. The inlet 114 is configured to receive air including suspended particulates P into the cyclone chamber 112. The shape of the cyclone chamber 112 produces a rotational (also called “cyclonic”) airflow within the cyclone chamber for the received air, and the received air is then fed out of the cyclone chamber 112 through the outlet 116. In some examples, the cyclone chamber 112 includes at least one outer wall 113 defining a conical, cylindrical, or other suitable shape for producing the rotational airflow in the cyclone chamber 112.

The rotational airflow in the cyclone chamber 112 produces centrifugal forces that drive or propel at least some suspended particulates P (including for example larger particulates, e.g., particulates having a diameter above 100 microns) radially outwardly toward the cyclone chamber outer wall(s) 113. The outwardly-driven particulates P may agglomerate or cluster together, and the clustered particulates P may then fall downwardly (due to gravity) along the cyclone chamber outer wall(s) 113 to the particulate removal system 130. The general flow path of particulates P radially outwardly toward the cyclone chamber outer wall(s) 113, where they become clustered and fall downward toward the particulate removal system 130, is indicated by arrows PCF. Thus, the cyclone filter 110 is configured to facilitate a rotational airflow within the cyclone chamber to remove at least a portion of the particulates P from the received air.

As shown in FIG. 1, the particulate removal system 130 may comprise a particulate reservoir 132 configured to collect and store particulates P removed from the cyclone chamber 112. Alternatively, the particulate removal system 130 may be a conduit 136 for delivering particulates P back into the environment (e.g., in a direction away from the cyclone chamber inlet 114) or to a remove reservoir, for example.

As noted above, the electrostatic filtration system 120 further facilitates the removal of particulates P from the air in the cyclone filter 110, especially small particulates (e.g., having a diameter below 100 microns, below 20 microns, or even below 10 microns, for example including particulates with diameters in the range of 2.5-10 microns). The electrostatic filtration system 120 comprises electrostatic filtration system electronics 121 including a power source 122, a microcontroller 124, and a separator 126. The power source 122 may comprise a battery (e.g., where the air treatment system 100 is embodied as a wearable or portable system) or a power cord for connection to an electrical grid.

The separator 126 includes (a) a first terminal 126a conductively connected to interior surface(s) 115 of the cyclone chamber outer wall(s) 113 and a second terminal 126b conductively connected to surface(s) 133 (e.g. interior surfaces) of the particulate removal system 130 (e.g., particulate reservoir 132). The cyclone chamber interior surface(s) 115 and particulate removal system surface(s) 133 may be formed from, coated with, or lined with a conductive material, for example a metal.

The microcontroller 124 is configured to control the separator 126 to:
(a) apply a first voltage with a first polarity (e.g., a negative voltage) to the conductive cyclone chamber interior surface(s) 115, to thereby charge the particulates P in the cyclone chamber 112 with the first polarity (e.g., resulting in negatively-charged particulates P), and
(b) apply a second voltage with an opposite second polarity (e.g., positive voltage) to the conductive particulate removal system surface(s) 133.

As a result of the voltages applied by the separator 126, the charged particulates P (e.g., negatively-charged particulates P) in the cyclone chamber 112 become electrostatically attracted to the particulate removal system 130 having the opposite charge as the particulates P (e.g., positively-charged surface(s) 133 of the particulate removal system 130). This electrostatic attraction creates an acceleration of the charged particulates P toward the particulate removal system 130, as indicated by arrows PEF. In other words, the electrostatic attraction pulls the charged particulates P toward the particulate removal system 130.

The electrostatic attraction provided by the electrostatic filtration system 120 may be particularly useful for removing particulates P from the cyclone filter 110 that are not effectively removed by the operation of the cyclone chamber 112 discussed above, for example small or low-mass particulates P that are insufficiently affected by centrifugal forces associated with the rotational airflow in the cyclone chamber 112 (discussed above) to be driven outwardly, cluster with other particulates P, and fall downwardly due to gravity to the particulate removal system 130 (e.g., particulates that follow a path generally indicated by arrows PCF).

Electrostatic filtration system electronics 121 may apply any suitable voltage polarities and magnitudes to conductive cyclone chamber interior surface(s) 115 and conductive particulate removal system surface(s) 133 to create an electrostatic attraction of particulates P to the particulate removal system 130.

For example, regarding polarity, in some examples electrostatic filtration system electronics 121 (e.g., microcontroller 124 and separator 126) may (a) apply a negative voltage to the conductive cyclone chamber interior surface(s) 115, to charge the particulates P in the cyclone chamber 112 with a negative charge, and (b) apply a positive voltage to the conductive particulate removal system surface(s) 133, to thereby attract the negatively-charged particulates P. In other examples, the electrical polarities may be reversed, which provides a similar electrostatic attraction of particulates P toward the particulate removal system 130. In other words, in some examples electrostatic filtration system electronics 121 may apply a positive voltage to the conductive cyclone chamber interior surface(s) 115 (to thereby positively charge the particulates P in the cyclone chamber 112) and apply a negative voltage to the conductive particulate removal system surface(s) 133, to thereby attract the positively-charged particulates P.

Regarding magnitude, in some examples electrostatic filtration system electronics 121 may apply a first negative voltage in the range of 5-10 kV to the conductive cyclone chamber interior surface(s) 115 and a second voltage of 0V to the conductive particulate removal system surface(s) 133.

As discussed above, the cyclone chamber interior surface(s) 115 and particulate removal system surface(s) 133 may be formed from, coated with, or lined with a conductive material, for example a metal, such that surfaces 115 and 133 may be charged by electrostatic filtration system electronics 121. In some examples, cyclone chamber interior surface(s) 115 and/or particulate removal system surface(s) 133 may be formed from, coated with, or lined with a conductive material having anti-microbial or anti-virus properties, for example silver, copper, or a copper alloy (e.g., brass or bronze), without limitation.

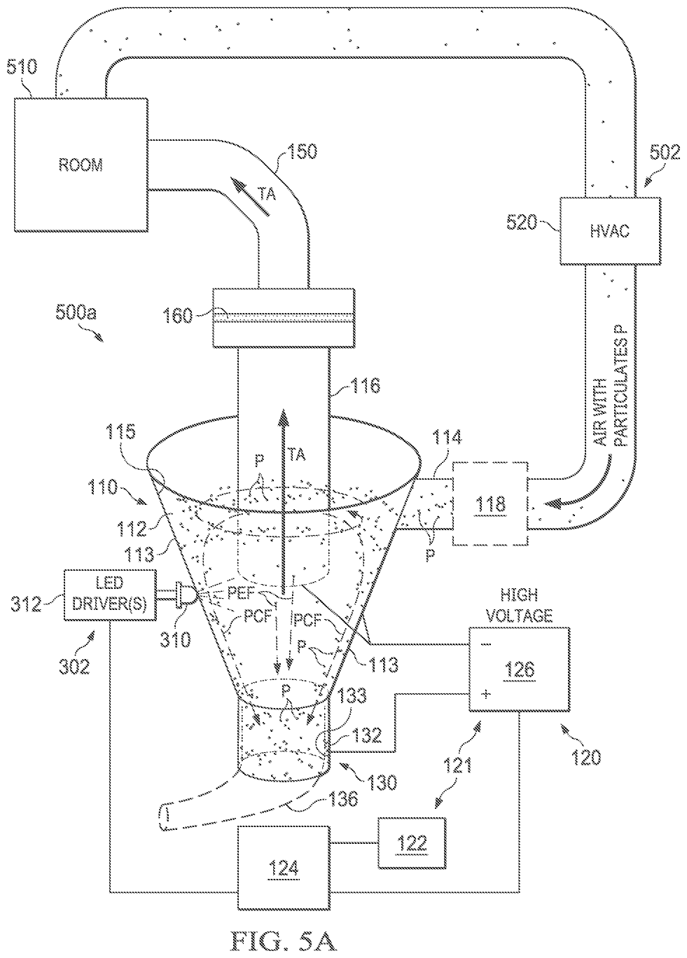

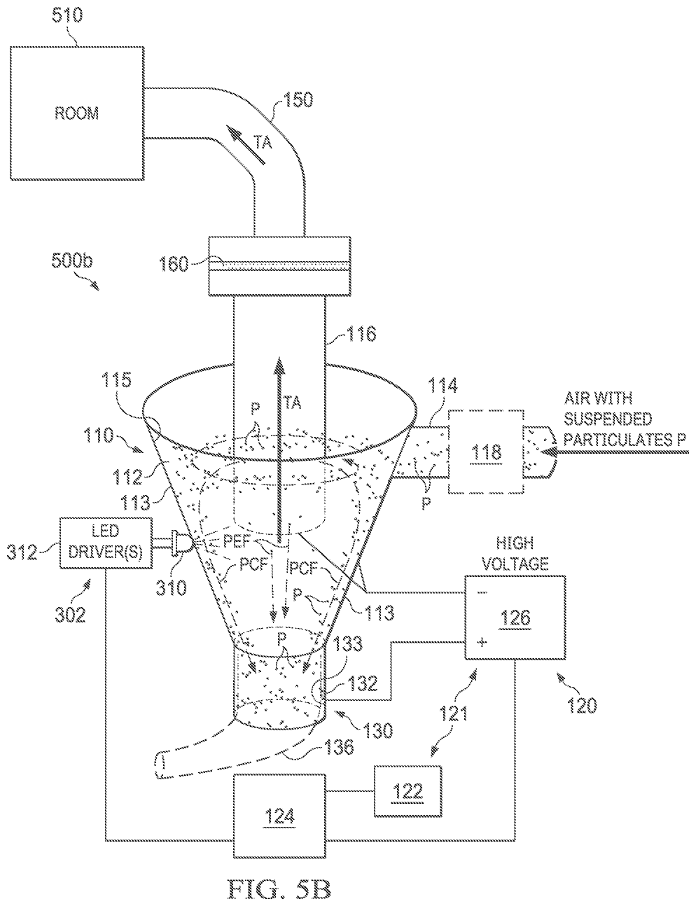

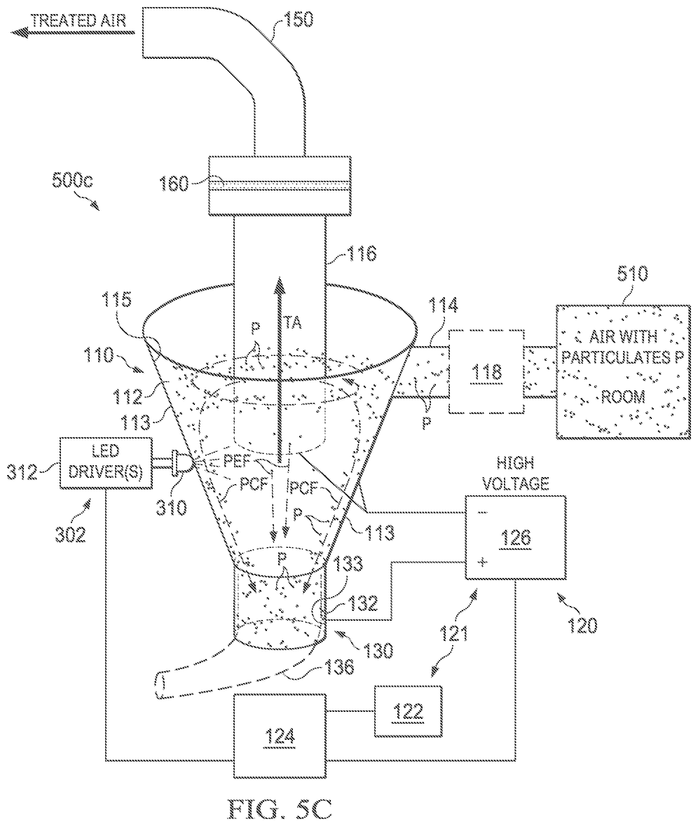

Referring to FIG. 1, after flowing (rotationally) through the cyclone chamber 112, wherein at least a portion of the particulates P are removed from the air by the cyclone filter 110 and electrostatic filtration system 120 as discussed above, the treated air TA flows out of the cyclone chamber 112 through outlet 116 to a respiratory interface 140 configured to present treated air TA for breathing by one or more person. In some examples, the respiratory interface 140 may comprise a facemask, a face shield including an air outlet, or any other device configured to deliver treated air TA into or near a person's mouth and/or nose. In other examples, e.g., the example shown in FIGS. 5A-5C discussed below, the respiratory interface 140 may comprise an outlet configured to deliver treated air TA to a room or other environment for breathing by one or more people.

In some examples, the respiratory interface 140 may connect the cyclone filter 110 with a user's respiratory system in a manner that transfers pressure generated by the user's respiratory system to the cyclone chamber 112, so that aspects of the user's breathing (e.g., inhalation and/or exhalation) generate or promote the rotational airflow in the cyclone chamber 112 for removing particulates P from the air received via the cyclone chamber inlet 114. For example, the respiratory interface 140 may comprise a mask configured to be sealed or partially sealed against a user's face (e.g., around the user's mouth, nose, or both). When the user inhales (inhalation), a negative pressure is generated in the user's respiratory system, which creates a suction force in the cyclone chamber 112 that generates a flow of air into the cyclone chamber inlet 114, around the cyclone chamber 112 (rotational airflow), and out through the cyclone chamber outlet 116 and to the respiratory interface, i.e. mask, 140.

In some examples, the air treatment system 100 include a blower (e.g., fan) 118 to generate or promote a positive pressure airflow into the cyclone chamber 112 via the cyclone chamber inlet 114, around the cyclone chamber 112 (rotational airflow), and through the cyclone chamber outlet 116 to the respiratory interface 140. For example, an air treatment system 100 wherein the respiratory interface 140 does not generate an airflow through the air treatment system 100 (e.g., where the respiratory interface 140 is not sealed against the user's face) may include the blower 118 to generate a positive pressure airflow through the air treatment system 100. As another example, where the respiratory interface 140 is configured to generate an airflow through the air treatment system 100 (e.g., where the respiratory interface 140 is at least partially sealed against the user's face), the blower 118 may be provided to promote or supplement the airflow through the air treatment system 100. In some examples, the blower 118 may be battery-powered, e.g., powered by a common power source, e.g., battery, 122 with the electrostatic filtration system 120 and/or other electronic components of air treatment system 100.

In some examples, at least one treated air delivery conduit 150 (e.g., at least one flexible or rigid hose, tube, or other conduit) may be connected between the cyclone chamber outlet 116 and respiratory interface 140. In some examples, a replaceable filter 160 may be arranged downstream of the cyclone filter outlet 116, for removing additional particulates P from the treated air TA. The replaceable filter 160 may comprise a cloth or cellulose filter cartridge, or other type of replaceable air filter. Because the cyclone filter 110 and electrostatic filtration system 120 may remove a substantial quantity particulates P upstream of the replaceable filter 160, replaceable filter 160 may receive substantial fewer particulates P as compared with many conventional replaceable filters. Thus, the replaceable filter 160 may allow a lower replacement frequency than such conventional replaceable filters.

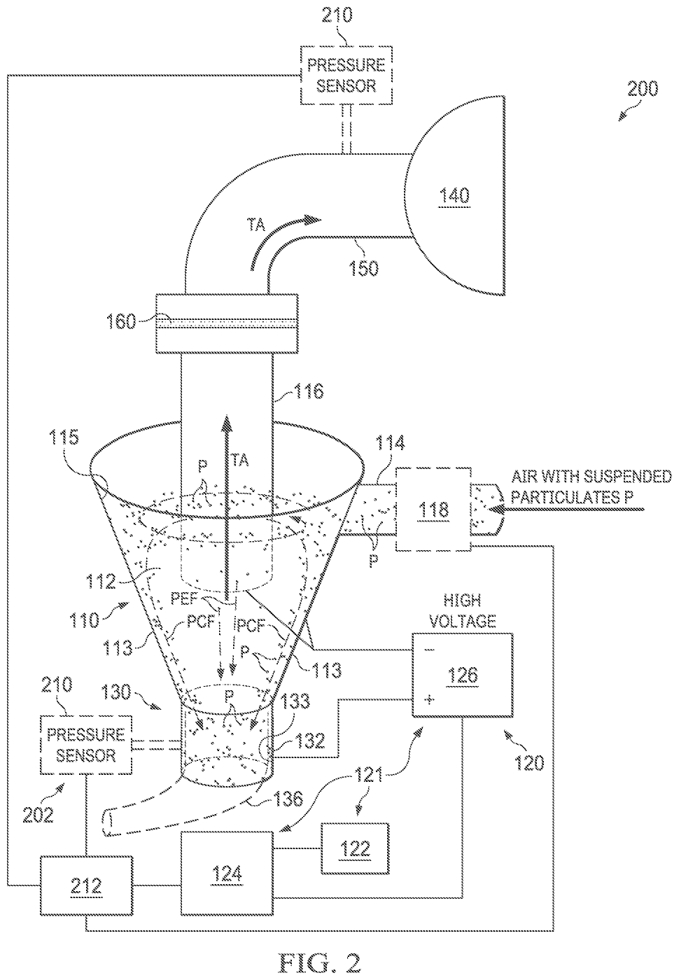

FIG. 2 shows an example air treatment system 200 according to one example. Air treatment system 200 may be similar to air treatment system 100 shown in FIG. 1, and also includes a pressure-based control system 202 configured to control at least one operational aspect of the air treatment system 200 based on a detected pressure or defined pressure change. The pressure-based control system 202 may include at least one pressure sensor 210 configured to monitor an air pressure at one or more location in the air treatment system 200. In one example the pressure-based control system 202 may maintain a running average using an analog integrator on the incoming sensor signal, and the integrator output may connect to a comparator. The other input of the comparator may connect directly to the input from the sensor(s) 210. When the input suddenly drops, the slow moving integrator output provides the threshold and the straight sensor input would trigger the detection

In one example the pressure-based control system 202 may use an analog integrator to maintain a running average of pressure signals from pressure sensor(s) 210, and the integrator output may be passed to a comparator. The other input of the comparator may connect directly to the input from the sensor(s) 210. When the sensor signals suddenly drop, the slow moving integrator output provides the threshold and the straight sensor input will trigger the defined pressure detection.

FIG. 2 shows two example locations of pressure sensor 210, namely a first example location in the particulate reservoir 132 and a second example location at the treated air delivery conduit 150 connected between the cyclone chamber outlet 116 and the respiratory interface 140.

The pressure-based control system 202 also includes pressure-based control electronics 212 configured to dynamically control (a) the electrostatic filtration system 120 via a connection to microcontroller 124 and/or (b) the blower 118 as a function of the air pressure monitored by pressure sensor(s) 210. Pressure-based control electronics 212 may include a microcontroller or other processor and control logic stored in memory. In some examples, pressure-based control electronics 212 may be configured to automatically detect inhalation events and/or exhalation events based on the pressure measurements from pressure sensor(s) 210. Types of inhalation events may include a start of inhalation, an end of inhalation, and an occurrence of an inhalation; and types of exhalation events may include a start of exhalation, an end of exhalation, and an occurrence of an exhalation. In one example, pressure-based control electronics 212 may detect inhalation events and/or exhalation events (e.g., start of inhalation, end of inhalation, occurrence of an inhalation, start of exhalation, end of exhalation, and/or occurrence of an exhalation, without limitation), for example by comparing the pressure measurements from pressure sensor(s) 210 to one or more stored threshold pressure values and/or reference pressure data or patterns, or otherwise analyzing the pressure measurements.

With respect to the electrostatic filtration system 120, pressure-based control electronics 212 may be configured to automatically detect inhalation events and/or exhalation events, and dynamically control the electrostatic filtration system 120 in response to detected inhalation events and/or exhalation events. Dynamically controlling the electrostatic filtration system 120 may include activating or deactivating the electrostatic filtration system 120, or otherwise controlling the voltages applied to the conductive cyclone chamber interior surface(s) 115 and/or conductive particulate removal system surface(s) 133, e.g., by providing control signals to microcontroller 124 or by direct control of the separator 126 (connection not shown).

In some examples, pressure-based control electronics 212 may be configured to dynamically control the electrostatic filtration system 120 based on detected inhalation events, for example a start of inhalation, an end of inhalation, or the occurrence of an inhalation.

In one example, pressure-based control electronics 212 may detect inhalation events, e.g., a start of inhalation, an end of inhalation, or the occurrence of an inhalation, based on pressure measurements from pressure sensor(s) 210, e.g., by comparing such pressure measurements against defined threshold values and/or reference pressure data or patterns. Pressure-based control electronics 212 may be configured to automatically activate the electrostatic filtration system 120 upon detecting a defined inhalation event (e.g., a start of inhalation or an occurrence of an inhalation), and continue to operate the electrostatic filtration system 120 until detecting a “no-inhalation” period during which no inhalation event is detected for a defined no-inhalation threshold duration (e.g., 15 seconds), and in response deactivate the electrostatic filtration system 120, e.g., until a next inhalation event is detected. In this manner, pressure-based control electronics 212 may be configured to automatically activate and operate the electrostatic filtration system 120 only while the user is using the air treatment system 100 for breathing, which may extend battery life or otherwise reduce power consumption.

The no-inhalation threshold duration (e.g., 15 seconds) for triggering a deactivation of the pressure-based control electronics 212 may be set (and stored in pressure-based control electronics 212) based on a typical or average maximum duration between breaths. In one example, pressure-based control electronics 212 may provide a user interface for setting or adjusting the no-inhalation threshold duration, e.g., based on the particular user's breathing habits or preferences for automatic deactivation of the electrostatic filtration system 120.

In another example, pressure-based control electronics 212 may be configured to automatically detect each start of inhalation based on pressure measurements from pressure sensor(s) 210, activate the electrostatic filtration system 120 as a function of each detected start of inhalation (e.g., upon each detected start of inhalation), automatically detect each end of inhalation based on pressure measurements from pressure sensor(s) 210, and deactivate the electrostatic filtration system 120 as a function of each detected end of inhalation (e.g., upon each detected end of inhalation or upon a defined delay period (e.g., 1 second) after each detected end of inhalation), such that the electrostatic filtration system 120 is active only during the inhalation phase of each breath, which may extend battery life or otherwise reduce power consumption.

With respect to the blower 118, pressure-based control electronics 212 may be configured to automatically detect inhalation events and/or exhalation events, and dynamically control the blower 118 based on the detected inhalation events and/or exhalation events. Dynamically controlling the electrostatic filtration system 120 may include activating or deactivating the blower 118, or otherwise controlling the power applied to the blower 118, e.g., to control the pressure and/or airflow produced by the blower 118. For example, the blower 118 may be controlled to maintain a low positive pressure in the respiratory interface (e.g., mask) 140, automatically adjusting for pressure loss caused by leaks.

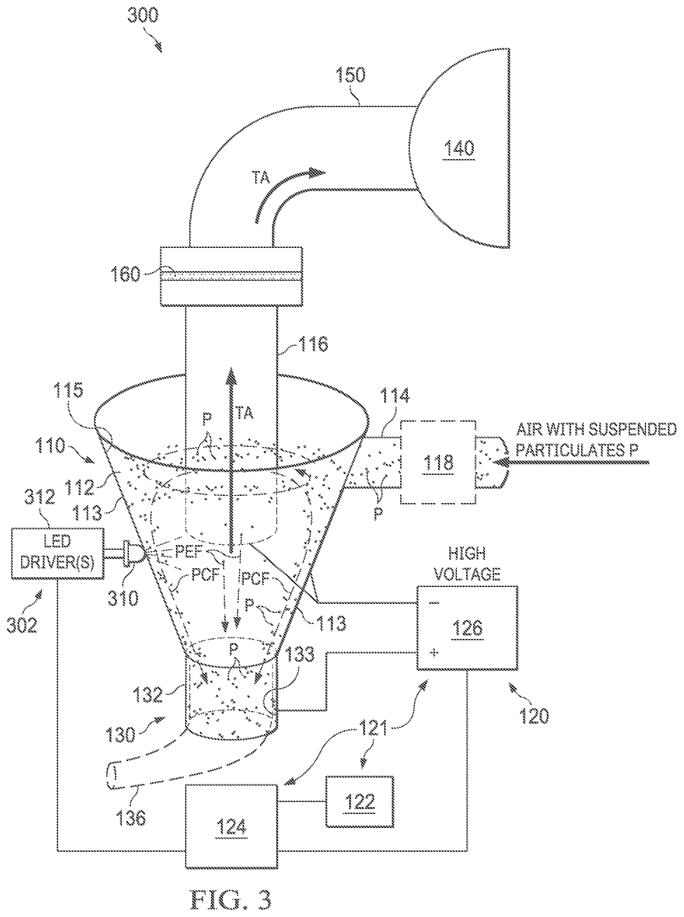

FIG. 3 shows an example air treatment system 300 according to one example. Air treatment system 300 may be similar to air treatment system 100 shown in FIG. 1, and also includes an ultraviolet purification system 302 configured to deliver ultraviolet radiation into the cyclone chamber 112 to affect particulates P in the cyclone chamber 112. As shown, ultraviolet purification system 302 may include ultraviolet LEDs 310 and corresponding LED drivers 312.

In some examples, ultraviolet purification system 302 may deliver ultraviolet C (UVC) radiation to the cyclone chamber 112 to affect organic particulates included in the particulates P in the received air within the cyclone chamber 112, for example to kill, destroy, or otherwise modify bacteria or virus particulates in the cyclone chamber 112. Some or all of such organic particulates may also be removed from the air and delivered to the particulate removal system 130 by the operation of the cyclone filter 110 and/or electrostatic filtration system 120 discussed above. LED drivers 312 may be responsive to microcontroller 124, which microcontroller 124 may provide control signals for LED drivers 312. LED drivers 312 may be provided power from power supply 122 (connection not shown).

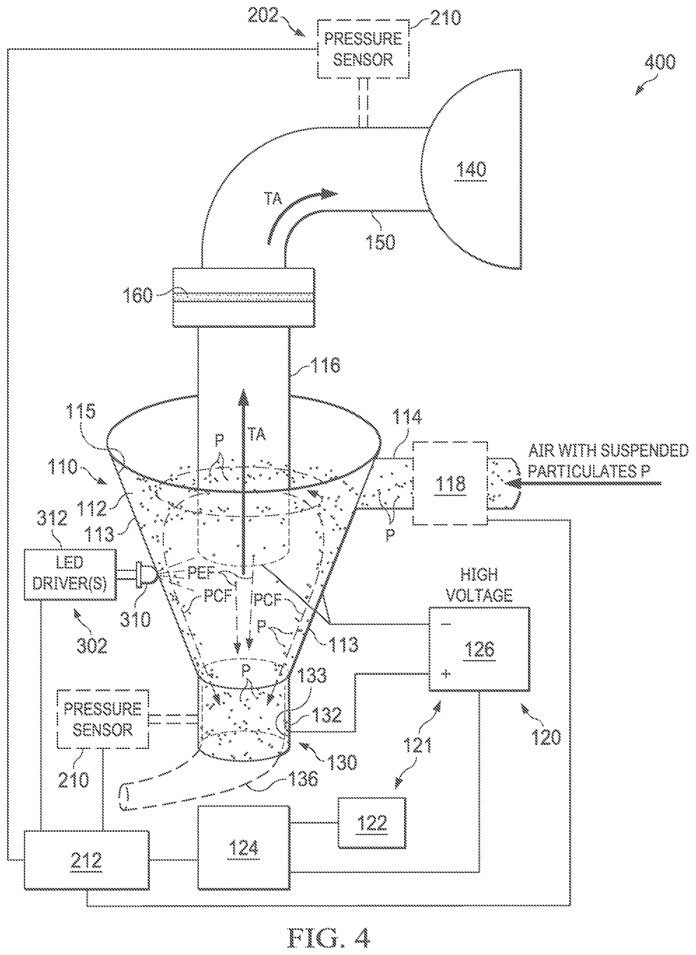

FIG. 4 shows an example air treatment system 400 according to one example. Air treatment system 400 may be similar to air treatment system 100 shown in FIG. 1, and also includes the pressure-based control system 202 as described above regarding FIG. 2 and the ultraviolet purification system 302 as described above regarding FIG. 3. Pressure-based control electronics 212 may be configured to dynamically control (a) the electrostatic filtration system 120, (b) the ultraviolet purification system 302, and/or (c) the blower 118, each as a function of the air pressure monitored by pressure sensor(s) 210.

In some examples, pressure-based control electronics 212 may be configured to (a) automatically detect inhalation events and/or exhalation events based on the pressure measurements from pressure sensor(s) 210 and (b) control both the electrostatic filtration system 120 and the ultraviolet purification system 302 based on the detected inhalation events and/or exhalation events. For example, pressure-based control electronics 212 may be configured to automatically detect each start of inhalation based on pressure measurements from pressure sensor(s) 210, activate the electrostatic filtration system 120 and ultraviolet purification system 302 as a function of each detected start of inhalation, automatically detect each end of inhalation based on pressure measurements from pressure sensor(s) 210, and deactivate the electrostatic filtration system 120 and ultraviolet purification system 302 as a function of each detected end of inhalation, such that the electrostatic filtration system 120 and ultraviolet purification system 302 are active only during the inhalation phase of each breath, which may extend battery life or otherwise reduce power consumption.

Any of the example air treatment systems discussed above may be embodied as a self-contained wearable system. For example, the cyclone filter 110, electrostatic filtration system 120, and ultraviolet purification system 302 (if present) may be arranged in a soft or rigid housing configured to be carried by a backpack, shoulder strap, chest strap, waist strap or belt, or secured to a user's shirt, pants, hat, helmet, or other article of clothing or wearable protective gear.

FIG. 5A shows an example air treatment system 500a connected to a heating ventilation and air conditioning (HVAC) system 502 for providing treated air to a room 510. The various components of air treatment system 500 may be similar to corresponding components of any of air treatment systems 100-400 discussed above. Although the air treatment system 500a is illustrated as being arranged downstream of the HVAC system 520, the air treatment system 500a may alternatively be arranged upstream of the HVAC system 520, or may be arranged within the HVAC system 520.

As shown, the HVAC system 520 may receive air from room 510 including suspended particulates P. The air treatment system 500 may receive the air with particulates P via inlet 114. The cyclone filter 110 and electrostatic filtration system 120 may remove particulates P from the air, and the ultraviolet purification system 302 may kill, destroy, or otherwise modify organic particulates P in the cyclone chamber 112, as discussed above. The filtered air exiting through outlet 116 may be delivered back to the room 510 via the treated air delivery conduit 150.

FIG. 5B shows an example air treatment system 500b similar to air treatment system 500a, but arranged for treating an external source of contaminated air and delivering the treated air to room 510. FIG. 5C shows an example air treatment system 500c similar to air treatment system 500a, but arranged for treating contaminated air in room 510 to deliver a flow of treated air outside the room 510.

1. An air treatment system, comprising:
a cyclone filter including:
a cyclone chamber;
an inlet configured to receive air including particulates into the cyclone chamber;
an outlet configured to output treated air from the cyclone chamber;
wherein the cyclone filter is configured to facilitate a rotational airflow within the cyclone chamber to remove at least a portion of the particulates from the received air; and
a particulate removal system configured to receive particulates removed from the received air by the cyclone filter; and
an electrostatic filtration system comprising electrostatic filtration system electronics configured to:
apply a first electric charge having a first polarity to the particulates in the cyclone chamber; and
apply a second electric charge having a second polarity opposite the first polarity to the particulate removal system;
such that the particulates in the cyclone chamber become charged with the first polarity and are electrostatically attracted to the particulate removal system.

2. The air treatment system of claim 1, wherein the electrostatic filtration system electronics are configured to:
apply the first electric charge having the first polarity to at least one conductive surface of the cyclone chamber to thereby apply the first electric charge to the particulates in the cyclone chamber; and
apply the second electric charge having the second polarity opposite the first polarity to at least one conductive surface of the particulate removal system.

3. The air treatment system of claim 2, wherein the at least one conductive surface of the cyclone chamber comprises silver or copper.

4. The air treatment system of claim 1, further comprising:
a pressure sensor configured to monitor an air pressure; and
pressure-based control electronics configured to dynamically control the electrostatic filtration system as a function of the monitored air pressure.

5. The air treatment system of claim 4, wherein the pressure-based control electronics are configured to dynamically control, based on the monitored air pressure, at least one of (a) a first voltage having the first polarity to at least one conductive surface of the cyclone chamber or (b) a second voltage having the second polarity to at least one conductive surface of the particulate removal system.

6. The air treatment system of claim 4, wherein the pressure-based control electronics are configured to:
automatically detect inhalation events based on the monitored air pressure, the automatically detected inhalation events including at least one of a start of inhalation, an end of inhalation, or an occurrence of an inhalation; and
automatically control the electrostatic filtration system based on the detected inhalation events.

7. The air treatment system of claim 6, wherein the pressure-based control electronics are configured to:
automatically detect an inhalation event based on the monitored air pressure;
automatically activate the electrostatic filtration system in response to the detected inhalation event;
automatically detect a no-inhalation period during which no inhalation event is detected for a defined no-inhalation threshold duration; and
automatically deactivate the electrostatic filtration system in response to the detected no-inhalation period.

8. The air treatment system of claim 4, wherein the control electronics are configured to:
detect a start of inhalation by a user based on the monitored air pressure;
activate the electrostatic filtration system as a function of the detected start of inhalation;
detect an end of inhalation by the user based on the monitored air pressure; and
deactivate the electrostatic filtration system as a function of the detected end of inhalation.

9. The air treatment system of claim 1, further comprising an ultraviolet purification system configured to deliver ultraviolet radiation to the cyclone chamber to affect at least some of the particulates in the received air.

10. The air treatment system of claim 9, wherein the ultraviolet purification system is configured to deliver ultraviolet C (UVC) radiation to the cyclone chamber to affect organic particulates in the cyclone chamber.

11. The air treatment system of claim 9, further comprising:
a pressure sensor configured to monitor an air pressure; and
pressure-based control electronics configured to control at least one of the electrostatic filtration system or the ultraviolet purification system as a function of the monitored air pressure.

12. The air treatment system of claim 11, wherein the pressure-based control electronics are configured to dynamically control the delivery of ultraviolet radiation to the cyclone chamber based on the monitored air pressure.

13. The air treatment system of claim 9, further comprising:
a pressure sensor configured to monitor an air pressure; and
pressure-based control electronics configured to:
automatically detect inhalation events based on the monitored air pressure, the automatically detected inhalation events including at least one of a start of inhalation, an end of inhalation, or an occurrence of an inhalation; and
automatically control the ultraviolet purification system based on the detected inhalation events.

14. The air treatment system of claim 13, wherein the pressure-based control electronics are configured to:
automatically detect an inhalation event based on the monitored air pressure;
automatically activate the ultraviolet purification system in response to the detected inhalation event;
automatically detect a no-inhalation period during which no inhalation event is detected for a defined no-inhalation threshold duration; and
automatically deactivate the ultraviolet purification system in response to the detected no-inhalation period.

15. The air treatment system of claim 9, further comprising:
a pressure sensor configured to monitor an air pressure; and
pressure-based control electronics configured to:
detect a start of inhalation by a user based on the monitored air pressure;
activate the ultraviolet purification system as a function of the detected start of inhalation;
detect an end of inhalation by the user based on the monitored air pressure; and
deactivate the ultraviolet purification system as a function of the detected end of inhalation.

16. The air treatment system of claim 1, wherein the particulate removal system comprises a particulate repository configured to receive and store particulates removed from the cyclone chamber.

17. The air treatment system of claim 1, further comprising a replaceable filter arranged downstream of the cyclone filter outlet.

18. The air treatment system of claim 17, wherein the replaceable filter comprises a cloth or cellulose filter cartridge.

19. The air treatment system of claim 1, further comprising a blower configured to generate a positive pressure airflow through the cyclone filter.

20. The air treatment system of claim 1, further comprising a respiratory interface configured to connect the cyclone filter with a user's respiratory system so that the rotational airflow in the cyclone chamber is generated by an inhalation of the user.

21. The air treatment system of claim 1, wherein the respiratory interface comprises a facemask.

22. The air treatment system of claim 1, wherein the air treatment system is a self-contained wearable system.

23. The air treatment system of claim 1, wherein the air treatment system is configured for connection to a heating, ventilation, and air conditioning (HVAC) system.

24. The air treatment system of claim 1, wherein the cyclone filter is configured to produce a rotational airflow in the cyclone chamber to propel at least a portion of the particulates in the cyclone chamber radially outwardly, resulting in particulate clusters that fall downwardly toward the particulate removal system.

25. An air treatment system for treating contaminated air, the air treatment system comprising:
a cyclone filter configured to receive air including particulates and produce a rotational airflow for removing at least some of the particulates;
an ultraviolet purification system configured to deliver ultraviolet radiation to the cyclone filter to kill or destroy organic particulates included in the particulates in the cyclone filter; and
an electrostatic filtration system configured to electrically charge the particulates in the cyclone filter to facilitate removal of particulates from the cyclone filter by electrostatic forces.

26. The air treatment system of claim 25, wherein the electrostatic filtration system comprises electronics configured to:
apply a first electric charge having a first polarity to the particulates in the cyclone filter; and
apply a second electric charge having a second polarity opposite the first polarity to a particulate removal system,
such that the particulates in the cyclone filter become charged with the first polarity and are electrostatically attracted to the particulate removal system.
